(19) United States
(12) Patent Application Publication
(10) Pub. No.: US 2026/0098251 A1
(43) Pub. Date: Apr. 9, 2026

(54) METHODS FOR NUCLEIC ACID EXTRACTION FROM TISSUES

(71) Applicant: Waters Technologies Corporation, Milford, MA (US)

(72) Inventor: Balasubrahmanyam Addepalli, Bellingham, MA (US)

(73) Assignee: Waters Technologies Corporation, Milford, MA (US)

(21) Appl. No.: 19/348,432

(22) Filed: Oct. 2, 2025

Related U.S. Application Data
(60) Provisional application No. 63/703,311, filed on Oct. 4, 2024.

(51) Int. Cl. C12N 15/10

(52) U.S. Cl. CPC C12N 15/1006

(57) ABSTRACT

Provided herein are methods for extracting nucleic acids from tissue samples. The methods can be used for the isolation of exogenous nucleic acids, including saccharide-conjugated nucleic acids, from a tissue sample. The methods allow for efficient extraction of the nucleic acids with compatibility for downstream analysis.

### CROSS-REFERENCE TO RELATED APPLICATION

This application claims priority from and the benefit of U.S. Application No. 63/703,311 filed on Oct. 4, 2024. The entire contents of this application are incorporated herein by reference.

### FIELD OF INVENTION

The present disclosure relates generally to methods for sample preparation from biological tissues. In particular, the present disclosure provides methods for extracting nucleic acids from a biological tissue for downstream analysis.

### BACKGROUND

Nucleic acid-based therapeutics, including gene therapies, require the accurate quantification and analysis of the nucleic acid payload. In particular with gene therapies, the quality control during manufacturing and development requires the extraction of nucleic acids from cells present in tissues, such as liver tissue samples. Said tissues pose challenges for extraction due to the presence of components such as extracellular matrix which can hinder extraction efficiency. Accordingly, there exists a need in the art for methods of extracting nucleic acids from tissue samples.

### SUMMARY OF INVENTION

Disclosed herein are methods for extracting nucleic acids from tissue samples. By utilizing multiple enzymatic digestion steps coupled with a physical disruption, the provided methods afford robust and highly efficient release of nucleic acids from tissue samples. Said nucleic acids, including exogenous nucleic acids (e.g., antisense oligonucleotides, short interfering RNA (siRNA), lipid conjugated oligonucleotides, locked nucleic acids etc.), can subsequently be analyzed using downstream analytical methods, such as liquid chromatography/mass spectrometry (LC/MS).

Accordingly, in one aspect disclosed herein is a method of extracting an exogenous nucleic acid from a biological tissue, the method comprising incubating the biological tissue with a collagenase, homogenizing the biological tissue with a physical homogenization method, incubating the biological tissue with a second protease, and extracting the exogenous nucleic acid with a solid phase extraction technique.

In some embodiments, the biological tissue is derived from liver, muscle, skin, cartilage, bone, tendon, ligament, heart, kidney, or intestine. In some embodiments, the physical homogenization method is bead homogenization, high pressure cell disruption, or sonication.

In some embodiments, the second protease is a serine protease or a metalloprotease. In some embodiments, the second protease is Proteinase K or trypsin.

In some embodiments, the exogenous nucleic acid is a saccharide-conjugated oligonucleotide. In some embodiments, the exogenous nucleic acid is a messenger RNA (mRNA), a small interfering RNA (siRNA), an anti-sense oligonucleotide (ASO), or a transfer RNA (tRNA).

In some embodiments, the step of adding a second protease comprises a first addition and a second addition of the second protease. In some embodiments, the second addition of the second protease occurs at least 30 minutes after the first addition of the second protease. In some embodiments, the second addition of the second protease occurs at least 60 minutes after the first addition of the second protease.

In some embodiments, the method further comprises incubating the biological tissue with a ribonuclease inhibitor (RNase Inhibitor).

In some embodiments, the solid phase extraction technique is a weak anion exchange technique.

### BRIEF DESCRIPTION OF THE DRAWINGS

FIG. 1A provides a schematic workflow of a sample preparation method according to an embodiment of the technology.

FIG. 1B provides another schematic workflow of another sample preparation method according to another embodiment of the technology.

### DETAILED DESCRIPTION

Disclosed herein are methods for extracting nucleic acids from a sample tissue, including saccharide-conjugate nucleic acids. The provided methods utilize a series of protease incubation steps coupled with physical disruption methods to afford high release levels of the target exogenous nucleic acids. The resultant exogenous nucleic acids released from the biological tissue can be subsequently extracted using a solid phase extraction (SPE) method, such as weak anion exchange SPE.

It should be noted that whenever a value or range of values of a parameter are recited, it is intended that values and ranges intermediate to the recited values are part of this disclosure. The word “about” if not otherwise defined means ±5%. It is also to be noted that as used herein and in the claims, singular forms of “a”, “an”, and “the” include plural references unless the context clearly dictates otherwise. Additional terms are defined throughout the specification.

### Extraction Methods

In one aspect, disclosed herein are methods for extracting exogenous nucleic acids from a biological tissue. Biological tissues contain extracellular matrix (ECM) that provides structural support to surrounding cells present in the technology. The ECM, when intact, impacts the ability to extract nucleic acids from cells present in the biological tissue. Efficient extraction of material, such as nucleic acids, from cells present in a biological tissue first requires the removal of ECM. While the components of the ECM vary based on tissue type, collagen is the most abundant protein within the ECM and constitutes the primary structural component of the ECM (Frantz et al., J Cell Sci, 2010, 123 (24): 4195-4200). Further examples of ECM proteins include elastins, actins, and tubulins.

### Collagenases

Accordingly, the presently disclosed methods utilize an incubation step with a collagenase. Collagenases are enzymes that break the peptide bonds in collagen, resulting in the degradation of collagen. By incubating a biological tissue with a collagenase, the collagen in the ECM is degraded. In vertebrates, there are at least 28 unique collagen molecules that can be formed, with types I, III, IV, and VII being the most prominent in mammals (Wu et al., Collagenases and their inhibitors: a review. Collagen and Leather (2023) 5:19). Certain collagenase enzymes have broad or narrow specificity to different collagenase types. Thus, selection of the collagenase enzyme is dependent on the ECM composition of the biological tissue (i.e., the collagen types present in said ECM).

A number of collagenase enzymes are known in the art, including those of the matrix metalloprotease (MMP) family. Non-limiting examples of collagenases include MMP-1, MMP-8, MMP-13, MMP-18, MMP-2, MMP-9, MMP-3, MMP-10, MMP-7, MMP-26, MMP-14, MMP-15, MMP-16, MMP-25, MMP-12, MMP-19, and MMP-20.

MMP-1 degrades collagen types I, II, III, VII, VIII, and X.

MMP-8 degrades collagen types I, II, III, V, VII, VIII, and X.

MMP-13 degrades collagen types I, II, III, and IV.

MMP-18 degrades collagen types I, II, III, and IV.

MMP-2 degrades collagen types I, II, III, IV, V, VII, and XI.

MMP-9 degrades collagen types IV, V, VII, X, and XIV.

MMP-3 degrades collagen types II, III, IV, IX, X, and XI.

MMP-10 degrades collagen types III, IV, and V.

MMP-7 degrades collagen types IV and X.

MMP-26 degrades collagen type IV.

MMP-14 degrades collagen types I, II, and III.

MMP-15 degrades collagen type I.

MMP-16 degrades collagen type I.

MMP-25 degrades collagen type IV.

MMP-12 degrades collagen type IV.

MMP-19 degrades collagen type I and IV.

MMP-20 degrades collagen type V.

Further examples of collagenases include Collagenase A, Collagenase B, Collagenase D, Collagenase H, Collagenase P. In some embodiments, a collagenase-like enzyme (e.g., a gelatinase, a dispase, or a liberase) could be used in place of, or in addition to, a collagenase.

While the collagen composition in an ECM varies by tissue type, collagens type I, II, and III constitute at least 80% of overall collagen in mammalian tissues. For example, collagen types I and III are often co-distributed and are widespread across tissue types. Collagen IX and II are often co-distributed and found in connective tissues. Collagens XI, XXIV, XXVII, XII, XIV, XX, and X are primarily found in tendons and cartilage. Collagens XIII and XVII are primarily found in epithelial cells. Collagens IV, VII, XV, XVII, XIX are primarily found in bone marrow (see, e.g., Karamanos et al., “A guide to the composition and functions of the extracellular matrix” (2021) The FEBS Journal Vol 288 (24): 6850-6912).

As such, selection of a particular collagenase is dependent on the biological tissue from which nucleic acids are being extracted. With respect to liver tissue, the ECM of healthy liver tissue primarily comprises collagen types I, III, IV, and V (Bedossa and Paradis “Liver Extracellular Matrix in Health and Disease” Journal of Pathology (2003) 200:504-515). Accordingly, one or more collagenases that degrade collagen types I, III, IV, and/or V would be selected for a liver tissue sample, including one or more the collagenases as described above. The collagen composition of ECM for different biological tissues are known in the art, and thus the appropriate collagenase enzymes can be selected based on the known collagen composition.

The appropriate concentration of the one or more collagenases may be selected based on the target tissue and cell type. In some embodiments, the collagenase has a concentration between 10 mg/mL to 200 mg/mL. In some embodiments, the collagenase has a concentration of between 10-50 mg/mL, 50-100 mg/mL, 100-150 mg/mL, or 150-200 mg/mL.

### Physical Homogenization

To aid in the disruption of the biological tissue, a step of physical homogenization is utilized. Physical homogenization uses a direct physical force to break down tissue and cells separate from an enzymatic or chemical process. Examples of physical homogenization include, but are not limited to, bead homogenization, sonication, high pressure cell disruption, and cryopulvarization.

Bead homogenization utilizes small beads with high force that shear tissues and/or cells which come into contact with the beads. Bead size and the speed of homogenization is selected based on the target tissue/cells. In some embodiments, the small beads are comprise a bead material. In some embodiments, the bead material comprises glass, ceramic, or zirconium.

Sonication utilizes ultrasonic sound waves to disrupt tissues and/or cells with shear force. The frequency, duration, and wattage of sonication is selected based on the target tissues/cells.

High pressure cell disruption, such as French press or cell press disruption methods, involves the passage of a tissue or cell sample through a narrow valve under high pressure via a piston. Passage via the narrow valve provides shear stress and the resultant disruption. The pressure is selected based on the target tissues/cells.

Cryopulvarization (also referred to as cryogrinding) involves freezing the biological tissue to cryogenic temperatures (e.g., temperatures below −150° C., temperatures below −160° C., temperatures below −170° C., temperatures below −180° C., temperatures below −190° C., temperatures below −200° C., etc.) decreasing the ductility of the biological tissue, enabling mechanical griding (e.g., using a physical homogenization technique described herein) of the biological tissue. Crypulvarization may be used to physically homogenize particularly soft biological tissue.

The appropriate physical homogenization method can be selected based on the target tissue and cell type. The physical homogenization method may selected based on the mechanical strength of the biological tissue.

### Proteases

To aid in the extraction of the nucleic acids from the cells present in the biological tissue, a second protease step is utilized. Preferably, the second protease has broad substrate specificity (i.e., is not specific to a certain protein). For example, but not by way of limitation, the second protease may be a serine protease or a metalloprotease.

Serine proteases are proteases that cleave peptide bonds via a catalytic triad or catalytic dyad mechanism. Examples of serine proteases include, but are not limited to:
trypsin, which cleaves a peptide bond following a positively charged amino acid;
chymotrypsin, with specificity to medium or large hydrophobic residues;
elastase, with specificity to small residues; and
proteinase K, which cleaves a peptide bond adjacent to carboxyl groups of aliphatic and aromatic amino acids.

Metalloproteases are proteases that cleave peptide bonds via a catalytic mechanism involving a metal, such as zinc or cobalt. Metalloproteases are known in the art and include those described above, such as collagenase. Further exemplary metalloproteases include DNases (particularly DNase I), dispases (particularly dispase II), hyaluronidases, and elastases.

In some embodiments, the second protease is proteinase K or trypsin.

To improve efficiency of the second protease step, the second protease may be added sequentially as two or more additions. The multiple additions of the second protease can improve the overall efficiency and mitigate the loss of enzymatic activity due to autoproteolysis of the protease.

For example, but not by way of limitation, a first amount of the second protease may be added to the sample, and a second amount of the second protease may be added following the first amount. The second amount may be added 30 minutes, 60 minutes, 90 minutes, 120 minutes, 150 minutes, 180 minutes, or more following the first amount. The first and second amounts of the protease may be at the same concentration or at different concentrations. The appropriate concentration of the second protease may be selected based on the tissue and cell type.

### Tissues and Exogenous Nucleic Acids

The methods provided herein are used in the extraction of exogenous nucleic acids from a biological tissue. Examples of biological tissues include those derived from liver, muscle, skin, cartilage, bone, tendon, ligament, heart, kidney, or intestine.

In one aspect, the biological tissue comprises one or more cells comprising an exogenous nucleic acid. The exogenous nucleic acid may be a messenger RNA (mRNA), an antisense oligonucleotide (ASO), a transfer RNA (tRNA), or a small interfering RNA (siRNA). The exogenous nucleic acid may be conjugated to one or more saccharides, thereby forming a saccharide-conjugated nucleic acid.

Extraction of the exogenous nucleic acids may be monitored using methods known in the art, including quantification or analysis with high performance liquid chromatography (HPLC), ultraviolet spectroscopy, mass spectrometry, or combinations thereof.

In some embodiments, the methods provided herein result in the extraction of at least 80%, at least 85%, at least 90%, or at least 95% of the exogenous nucleic acid present in a biological tissue sample. The efficiency of an extraction may be measured by adding a reference oligonucleotide (e.g., a known amount of a reference oligonucleotide) to a biological tissue sample prior to performing the extraction experiment. The recovery of both the reference oligonucleotide and the exogenous nucleic acid is then monitored, and the recovery of the reference oligonucleotide is compared to the known amount of the reference oligonucleotide.

To prevent degradation of the exogenous nucleic acids, one or more ribonuclease inhibitors, also known as RNase inhibitors, may be utilized. Ribonuclease inhibitors bind to ribonucleases, which degrade RNA.

To further aid in recovery, quantification, and analysis of the exogenous nucleic acids, the digested tissue sample may be treated with solid phase extraction to isolate the nucleic acids. Any solid phase extraction method that can preferentially enrich for nucleic acids is suitable for use in the present methods.

Examples of suitable solid phase extraction methods include, but are not limited to, weak anion exchange (WAX), hydrophilic adsorption, and reversed phase adsorption extraction methods. In some embodiments, the solid phase extraction is weak anion exchange. Hydrophilic adsorption methods are described in, for example, U.S. Pat. No. 11,597,789. Solid phase extraction sorbents, including weak anion exchange, are known in the art and are described in, for example, U.S. Pat. Nos. 11,161,057 and 11,376,561, incorporated herein by reference.

The resultant nucleic acids, following solid phase extraction, may further be analyzed using methods known in the art, including mass spectrometry, high performance liquid chromatography, ultraviolet spectroscopy, or combinations thereof.

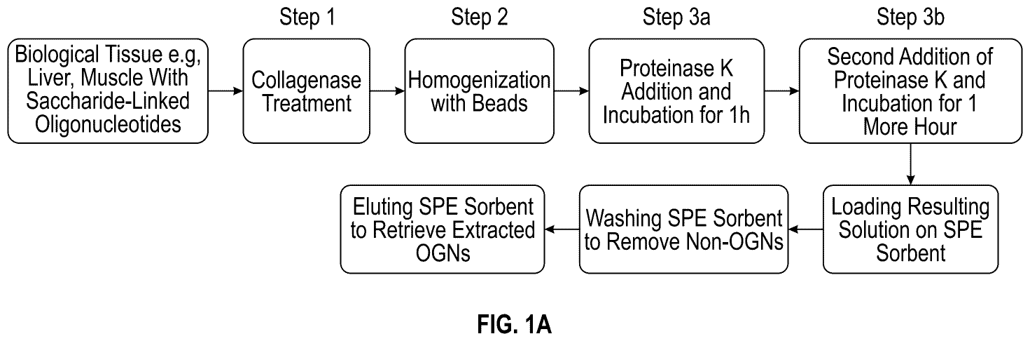

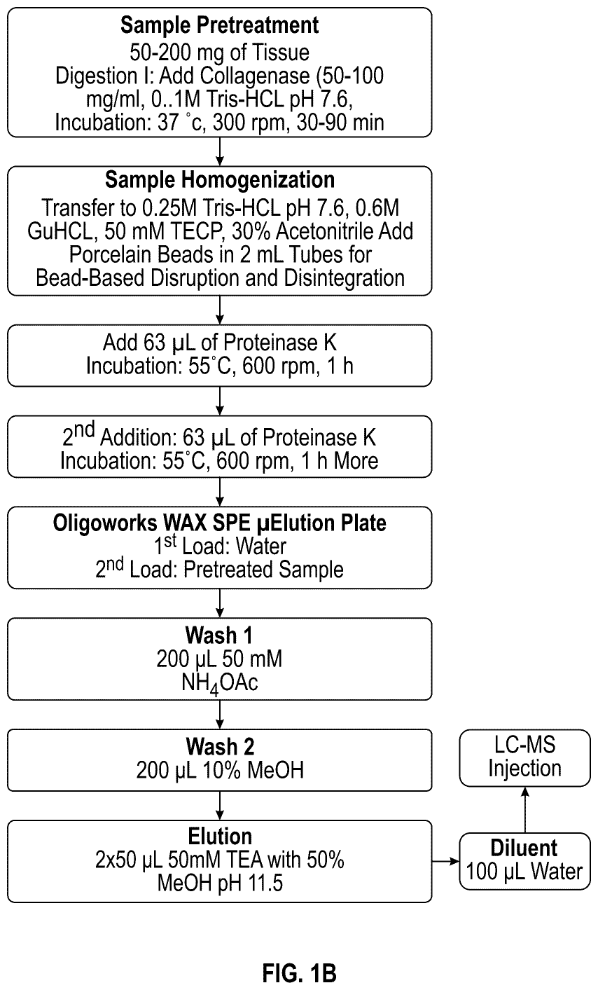

Example 1 and FIG. 1A and FIG. 1B provide exemplary workflows of the methods for extracting exogenous nucleic acids from a biological tissue.

### EXAMPLES

### Example 1: Extraction of Exogenous Nucleic Acids from a Biological Tissue

A method for extracting exogenous nucleic acids from a biological tissue is described. A biological tissue comprising an exogenous nucleic acid is first treated with a collagenase. The sample is then homogenized using a physical homogenization method, such as bead homogenization. A first amount of proteinase K is added and the sample is incubated for 1 hour. A second amount of proteinase K is added and the sample is incubated for 1 hour. The sample can then be loaded on a solid phase extraction sorbent. The sorbent is washed to remove unbound analytes. The target exogenous nucleic acids may then be eluted.

50-100 mg/mL of collagenase will be added to 50-200 mg of tissue in 0.1M Tris-HCl (pH 7.6). The sample will be incubated at 37° C. with shaking at 300 rpm for 30-90 minutes. Following the incubation step, the sample will be transferred to 0.25M Tris-HCl (pH 7.6), 0.6M guanidine hydrochloride, 50 mM TCEP, and 30% acetonitrile. Porcelain beads will be added to tubes containing the sample, such as Precellys® tubes (available from Bertin Technologies) and the sample is disrupted using bead homogenization. Following bead homogenization, 63 μL of RapiZyme™ Proteinase K Digestion Module (available from Waters Technology Corporation and contains Proteinase K) will be added and the sample incubated at 55° C. at 600 rpm for 1 hour. A second amount of 63 μL of RapiZyme™ Proteinase K Digestion Module will be added and the sample incubated at 55° C. at 600 rpm for 1 hour. The sample will then be loaded on a solid phase extraction WAX sorbent (OligoWorks WAX SPE, available from Waters Technologies Corporation, Milford MA). The sample will then be washed with 200 μL of 50 mM NH₄OAc, 200 μL of 10% MeOH, and eluted with 2×50 μL 50 mM TEA with 50% MeOH (pH 11.5). The eluted sample can be diluted with 100 μL of water and subsequently analyzed with LC-MS or another analytical technique.

### Example 2: Improved Recovery of Exogenous Oligonucleotides with Collagenase and Proteinase K

The present example is directed to a method of extracting an exogenous oligonucleotide from a liver tissue sample. First, 50 mg of a bovine liver tissue sample including GalNAc conjugated Oligonucleotide was pretreated with a 50 mg/L collagenase solution per mg of tissue for a collagenase digestion time at 37° C. with 600 rpm of mixing. Then, the sample was physically homogenized using bead homogenization.

Bead homogenization was performed in a 2 mL bead tube including 50 mg of treated sample and 500 μL of a homogenization mixture including 10 μL of reference oligonucleotide 100 mM ammonium acetate (pH 5.5 buffer), 115 μL of 1 M tris (hydroxymethyl)aminomethane hydrochloride (Tris-HCl; pH 7.5), 150 μL of MeCN, 50 μL of 0.5 M tris (2-carboxyethyl) phosphine hydrochloride (TCEP-HCl), 50 μL of 6M guanine hydrochloride, and 60 μL of the RapiZyme™ Proteinase K Digestion Module (available from Waters Technology Corporation). The beads were disrupted at 5800 rpm for 15s, followed by a 30s pause, and a second 15 second disruption at 5800 rpm.

The tissue sample was then extracted and stirred at 600 rpm and 55° C. for 1 hr. 65 μL of Rapizyme™ Proteinase K Digestion Module was then added to the tissue sample, and the sample was stirred for a proteinase K digestion time at 600 rpm and 55° C. The sample was then clarified with refrigerated microcentrifugation for 30 min at 10000 rpm, and purified using the OligoWorks™ WAX SPE Microplate Purification platform (available from Waters Technology Corporation). 100 μL of the homogenized supernatant was diluted with 100 μL of 50 mM sodium acetate buffer (pH 5.5), and the mixture was loaded onto the microplate purification platform. The mixture was washed twice with 200 μL of 100 mM sodium acetate (pH 5.5), then twice with 200 μL of 10% methanol in water. The sample was then eluted with 2 washes of 25 μL Oligoworks™ SPE Eluent (available from Waters Technology Corporation), and the eluate was diluted with an equal volume of water. The eluate was then analyzed with LC-MS to determine the extraction of GalNAc conjugated oligonucleotide.

The experiment was performed for a variety of collagenase treatment times and proteinase K treatment times, and compared to an experiment only using a proteinase K treatment step. The results are summarized in Table 1.

TABLE 1
Experimental Conditions for Collagenase
and Proteinase K Treatment

| | Collagenase | Proteinase | GalNAc Conjugated |
|---|---|---|---|
| | Treatment | K Treatment | Oligonucleotide |
| | Time | Time | Recovery |
| Experiment 1 | 0 | 1 × 2 hr | 35% |
| Experiment 2 | 30 min | 1 × 2 hr | 65% |
| Experiment 3 | 30 min | 2 × 1 hr | 75% |
| Experiment 4 | 60 min | 2 × 1 hr | 82% |

As shown in FIG. 3, each experiment including a collagenase treatment step and a proteinase K treatment step showed marked improvement over the proteinase K treatment alone. In particular, Experiment 3 and Experiment 4 recovered more than twice the GalNac Conjugated oligonucleotide compared to proteinase K alone.

1. A method of extracting an exogenous nucleic acid from a biological tissue, comprising:
a) incubating the biological tissue with a first protease, wherein the first protease is a collagenase;
b) homogenizing the biological tissue using a physical homogenization method;
c) incubating the biological tissue with a second protease; and
d) extracting the exogenous nucleic acid with solid phase extraction.

2. The method of claim 1, wherein the biological tissue is derived from liver, muscle, skin, cartilage, bone, tendon, ligament, heart, kidney, or intestine.

3. The method of claim 1, wherein the physical homogenization method is bead homogenization, high pressure cell disruption, or sonication.

4. The method of claim 1, wherein the second protease is a serine protease or a metalloprotease.

5. The method of claim 4, wherein the second protease is Proteinase K or trypsin.

6. The method of claim 1, wherein the exogenous nucleic acid is a saccharide-conjugated oligonucleotide.

7. The method of claim 1, wherein the exogenous nucleic acid is a messenger RNA (mRNA), a small interfering RNA (siRNA), an anti-sense oligonucleotide (ASO), or a transfer RNA (tRNA).

8. The method of claim 1, wherein step c) comprises a first addition and a second addition of the second protease.

9. The method of claim 8, wherein the second addition of the second protease occurs at least 30 minutes after the first addition of the second protease.

10. The method of claim 8, wherein the second addition of the second protease occurs at least 60 minutes after the first addition of the second protease.

11. The method of claim 1, further comprising incubating the biological tissue with a ribonuclease inhibitor (RNase Inhibitor).

12. The method of claim 1, wherein the solid phase extraction is weak anion exchange.
